(19) United States
(12) Patent Application Publication
(10) Pub. No.: US 2026/0183440 A1
(43) Pub. Date: Jul. 2, 2026

(54) HYDROGENE PEROXIDE AND STEAM STERILIZER

(71) Applicant: TUTTNAUER LTD., Beit Shemesh (IL)

(72) Inventors: Daniel RESHEF, Jerusalem (IL); Yuval SHILDERMAN, Ness Ziona (IL); Yossi SHENTAL, Hod Hasharon (IL); Haggai ABBO, Kiryat Ono (IL); Yona MAGEN, Nechusha (IL)

(21) Appl. No.: 18/867,825

(22) PCT Filed: May 23, 2023
(86) PCT No.: PCT/IB2023/055287

§ 371 Date: Nov. 21, 2024

Related U.S. Application Data
(60) Provisional application No. 63/344,685, filed on May 23, 2022.

(51) Int. Cl. A61L 2/208; A61L 2/07

(52) U.S. Cl. CPC A61L 2/208; A61L 2/07

(57) ABSTRACT

The present invention is of a sterilizer system for providing a steam sterilization cycle and a vaporized hydrogen peroxide sterilization cycle. The sterilizer system features a sterilization chamber for sterilizing at least one item, a system for providing steam sterilization to the sterilization chamber and a system for providing vaporized hydrogen peroxide sterilization to the sterilization chamber. In addition, the present invention is of methods of sterilization of at least one item with a steam sterilization cycle or a vaporized hydrogen peroxide sterilization cycle using the sterilizer system of the present invention.

This application claims priority from U.S. Provisional Patent Application Ser. No. 63/344,685 filed on May 23, 2022 and incorporated by reference herein in entirety.

### FIELD OF THE INVENTION

The present invention relates to a sterilizer. Moreover, the present invention is of a sterilizer, which provides both steam sterilization and sterilization with vaporized hydrogen peroxide.

### BACKGROUND OF THE INVENTION

Sterilization is routinely carried out on many different items to prevent infection and is essential for instruments such as surgical, medical, veterinary, cosmetic and dental tools. A commonly used sterilizer is an autoclave, which employs steam to sterilize items placed in its chamber. Steam sterilization is an effective technique and is reliable, non-toxic and economical. However, heat is detrimental to certain substances, which include a variety of polymers and adhesives, making steam sterilization not a viable option for heat sensitive materials. As such, for items constructed from heat sensitive materials, cold sterilization, or sterilization below certain temperatures is recommended.

One type of cold sterilizer uses vaporized hydrogen peroxide (VHP). Sterilization with vaporized hydrogen peroxide may be a useful way to sterilize heat sensitive items.

It would be advantageous for a user to have the option of using cold sterilization or hot sterilization, depending on the item to be sterilized. The purchase of two separate sterilizers is costly and bench space can be limited making it difficult to own and use two sterilizers. A single sterilizer which is configured to provide both steam and cold vaporized hydrogen peroxide sterilization would overcome these problems. However, such a combination is problematic, as at least some of the components and materials used in these two different methods are not compatible with both sterilization mediums and conditions.

It would be desirable to have a single sterilizer which could provide both steam sterilization and cold sterilization. It would be beneficial to have a system configured to overcome compatibility problems between the components of the two methods. It would also be advantageous if the hybrid system could be built so that the two sterilization methods use at least some joint components to reduce costs and the size of the hybrid system. The present invention provides such a system and methods of use thereof.

### SUMMARY

The invention may have several aspects. One aspect is a sterilizer system for providing a steam sterilization cycle and a vaporized hydrogen peroxide sterilization cycle. The sterilizer system may include a sterilization chamber for sterilizing at least one item. The sterilization system may include a system for providing steam sterilization to the sterilization chamber. The sterilization system may include a system for providing vaporized hydrogen peroxide sterilization to the sterilization chamber. The sterilization chamber may be a pressure vessel.

In various embodiments of the sterilizer system, the system may include at least one heater for heating the chamber. The system may include a vaporizer configured for vaporizing hydrogen peroxide from a source of hydrogen peroxide and for generating steam from water, the vaporizer for providing vaporized hydrogen peroxide or steam to the chamber. The system may include at least one pump for removing air from the chamber. The system may include at least one delivery system for delivering a flow of the liquid and vaporized hydrogen peroxide or flow of the water and steam in the sterilizer system. The system may include a bypass system for bypassing hydrogen peroxide sensitive components of the sterilizer system. The system may include a power source for powering the sterilizer system. The system may include a bypass system to bypass at least part of the system for providing vaporized hydrogen peroxide sterilization to the sterilization chamber. The system may include a bypass system to bypass at least part of the system for providing steam sterilization to the sterilization chamber. The sterilization chamber may be configured for sterilizing the at least one item with vaporized hydrogen peroxide or steam. The sterilization chamber may include a stainless steel chamber and a stainless steel door. The vaporizer may be an aluminum heat exchanger. The at least one pump may be a vacuum pump configured for use with steam and vaporized hydrogen peroxide. The at least one heater may include a heater attached to the periphery of the chamber. The at least one heater may include a heater attached to the door. The at least one heater may include a heater attached to the back wall of the chamber. The vaporizer may be a plurality of vaporizers and wherein one vaporizer may be configured for vaporizing hydrogen peroxide from a source of hydrogen peroxide and a second vaporizer may be configured for generating steam from water. The at least one pump may be a plurality of pumps and wherein one pump may be configured to be employed during a sterilization cycle with vaporized hydrogen peroxide and a second pump may be configured to be employed during a steam sterilization cycle.

In various embodiments of the system, the system for providing vaporized hydrogen peroxide sterilization to the sterilization chamber may include a source of liquid hydrogen peroxide. The at least one delivery system may feature channels for introducing a dose of liquid hydrogen peroxide into the sterilization system and for delivering the liquid and vaporized hydrogen peroxide in the sterilizer system. The system for providing vaporized hydrogen peroxide sterilization to the sterilization chamber may include a plurality of valves positioned about the channels. The plurality of valves may include at least two valves for drawing out a dose of the liquid hydrogen peroxide, by opening and closing of the valves to expose the dose to the pressure of the sterilization chamber. The plurality of valves may include at least one valve configured to introduce air at atmospheric pressure into the liquid hydrogen peroxide delivery channel to facilitate flow of the dose of liquid hydrogen peroxide into the vaporizer and the produced vaporized hydrogen peroxide into the sterilization chamber. The vaporizer may be configured to provide vaporized hydrogen peroxide to the sterilization chamber. The plurality of valves may be made from a heat resistant and hydrogen peroxide compatible material. The system for providing vaporized hydrogen peroxide sterilization to the sterilization chamber may include a bypass system to bypass at least part of the system for providing steam sterilization to the sterilization chamber. The bypass system may include at least one valve, which can be closed to block at least part of the steam sterilization system. The system may include at least one access control element to block or provide access to at least part of the at least one delivery system for delivering the liquid hydrogen peroxide. The at least one heater may include a heater attached to the periphery of the chamber. The at least one heater may include a heater attached to the door. The at least one heater may include a heater attached to the back wall of the chamber. The system for providing vaporized hydrogen peroxide sterilization to the sterilization chamber may include a trap disposed about the outlet of the at least one pump, wherein the at least one pump is for removing air from the chamber, the trap for filtering odors and remnants of hydrogen peroxide. The system for providing vaporized hydrogen peroxide sterilization to the sterilization chamber may include an access control element to block the filter during a steam sterilization cycle. The at least one delivery system of the system for providing vaporized hydrogen peroxide sterilization to the sterilization chamber may include a dosing pump for introducing a dose of liquid hydrogen peroxide from a source of liquid hydrogen peroxide into the sterilization system to the vaporizer and wherein the vaporizer is configured to provide vaporized hydrogen peroxide to the sterilization chamber.

In various embodiments of the system, the system for providing steam sterilization to the sterilization chamber may include a source of water. The at least one delivery system may feature channels for introducing water and for delivering the water and steam in the sterilizer system. The system for providing steam sterilization to the sterilization chamber may include a water pump for pumping the water through the channel/s. The system for providing steam sterilization to the sterilization chamber may include a plurality of valves positioned about the channels, which can be configured to block the channel or provide access to the channel. The system for providing steam sterilization to the sterilization chamber may include a heat exchanger for cooling the steam and hot air expelled from the chamber by the at least one air removing pump and wherein the expelled steam and hot air are cooled prior to reaching the at least one air removing pump. The vaporizer may be configured for generating steam for providing steam to the chamber. The system for providing steam sterilization to the sterilization chamber may include at least one access control element to block at least part of the at least one delivery system for delivering the liquid hydrogen peroxide. The at least one heater may include a heater attached to the periphery of the chamber. The system for providing steam sterilization to the sterilization chamber may include a heat exchanger bypass, which is configured to bypass the heat exchanger and protect it from hydrogen peroxide during a vaporized hydrogen peroxide sterilization cycle.

Another aspect is a method of steam sterilization of at least one item. The method may include inserting at least one item in the sterilization chamber of the sterilizer system as herein described. The method may include selecting a steam sterilization cycle. The method may include blocking at least part of the delivery system for delivering and flow of the liquid and vaporized hydrogen peroxide. The method may include heating the sterilization chamber. The method may include removing air from the sterilization chamber with the at least one pump, the at least one pump featuring a vacuum pump. The at least one pump may be a plurality of pumps, wherein a first pump is configured to be employed during a sterilization cycle with vaporized hydrogen peroxide and a second pump is configured to be employed during a steam sterilization cycle and the method may include removing air from the sterilization chamber with the second pump configured to be employed during a steam sterilization cycle. The method may include cooling the removed hot air and steam evacuated from the chamber. The method may include opening the delivery system for facilitating the flow of water in the system to the vaporizer. The method may include pumping water into the vaporizer to produce steam. The method may include introducing the steam into the evacuated sterilization chamber. The method may include sterilizing the at least one item with the steam.

In various embodiments of the method of steam sterilization the blocking may include closing a valve, which is adaptable to block or provide access to the hydrogen peroxide inlet channel. The heating the sterilization chamber may include heating the surfaces of the chamber with a jacket heater. Cooling the removed hot air may include providing the expelled air and steam with access to a heat exchanger and pumping the hot air and steam through the heat exchanger to cool it. Providing access to the heat exchanger may include opening at least one valve for facilitating opening a channel to the heat exchanger. The method may include drying the at least one item.

An additional aspect is a method of sterilization of at least one item with vaporized hydrogen peroxide. The method of sterilization of at least one item with vaporized hydrogen peroxide may include inserting at least one item in the sterilization chamber of the sterilizer system as described herein. The method may include selecting a vaporized hydrogen peroxide sterilization cycle. The method may include blocking at least part of the delivery system configured for delivering a flow of water and steam. The method may include heating the sterilization chamber and the at least one item to prevent hydrogen peroxide condensation. The method may include using the bypass system to block and bypass a heat exchanger for cooling air and steam in a steam sterilization cycle. The method may include removing air from the sterilization chamber with the at least one pump, the at least one pump featuring a vacuum pump. The at least one pump may be a plurality of pumps, wherein a first pump is configured to be employed during a sterilization cycle with vaporized hydrogen peroxide and a second pump is configured to be employed during a steam sterilization cycle and the method may include removing air from the sterilization chamber with the first pump configured to be employed during a vaporized hydrogen peroxide sterilization cycle. The method may include drawing a dose of liquid hydrogen peroxide through and into an inlet of the at least one delivery system for delivering liquid hydrogen peroxide. The method may include facilitating flow of the liquid hydrogen peroxide to the vaporizer. The method may include vaporizing the liquid hydrogen peroxide to produce vaporized hydrogen peroxide. The method may include introducing the vaporized hydrogen peroxide into the chamber. The method may include sterilizing the at least one item with the vaporized hydrogen peroxide.

In various embodiments of the method of sterilization of at least one item with vaporized hydrogen peroxide the sterilization system may include at least two valves for drawing out a dose of liquid hydrogen peroxide, and wherein drawing a dose may include opening and closing of the valves to expose the dose to the pressure of the sterilization chamber. The sterilization system may include at least one valve for introducing air to facilitate flow of the dose of liquid hydrogen peroxide into the vaporizer and the produced vaporized hydrogen peroxide into the chamber and wherein facilitating flow of the liquid hydrogen peroxide may include opening the valve to introduce the air at atmospheric pressure into the liquid hydrogen peroxide delivery channel. The sterilization system may include at least one trap and the method may include connecting the trap distally to the outlet of the pump to filter hydrogen peroxide.

A still further aspect is a method of converting an autoclave for steam sterilization to a system configured for providing a steam sterilization cycle and a hydrogen peroxide sterilization cycle. The method may include providing an autoclave. The autoclave may include a sterilization chamber for sterilizing at least one item. The autoclave may include a heater, which may feature a heating jacket surrounding the chamber for heating the chamber. The autoclave may include a vaporizer for generating steam for providing steam to the chamber. The autoclave may include a delivery system for delivering a flow of water and steam in the sterilizer system. The autoclave may include a vacuum pump for expelling air and steam from the chamber. The autoclave may include a power source for powering the autoclave. The method may include applying at least one bypass element, which is adaptable to block or provide access to the delivery system for delivering a flow of water and steam in the sterilizer system. The method may include applying a delivery system which may feature channels for delivering a flow of liquid and vaporized hydrogen peroxide in the sterilizer system. The method may include applying at least one bypass element, which is adaptable to block or provide access to the delivery system for liquid and vaporized hydrogen peroxide. The method may include connecting the hydrogen peroxide delivery system to the vaporizer for generating vaporized hydrogen peroxide in a vaporized hydrogen peroxide sterilization cycle.

In various embodiments of the method of converting an autoclave for steam sterilization to a system configured for providing a steam sterilization cycle and a hydrogen peroxide sterilization cycle, the method may include applying a heater on the door of the chamber. The method may include applying a heater on the back wall of the chamber. The applying at least one bypass element may include applying at least one valve. The applying a delivery system may include applying a plurality of valves positioned about the channels. The plurality of valves may include at least two valves for drawing out a dose of the liquid hydrogen peroxide, by opening and closing of the valves to expose the dose to the pressure of the chamber. The plurality of valves may include at least one valve for introducing air at atmospheric pressure to facilitate flow of the dose of liquid hydrogen peroxide into the vaporizer and the produced vaporized hydrogen peroxide into the chamber. The method may include attaching a trap adjacent to a distal end of an outlet of the vacuum pump to filter the hydrogen peroxide. Providing an autoclave may include providing the vacuum pump constructed from heat and hydrogen peroxide resistant materials.

### BRIEF DESCRIPTION OF THE DRAWINGS

The various features of the invention will best be appreciated by simultaneous reference to the description which follows and the accompanying drawings, which are not drawn to scale and in which:

FIG. 1 shows a schematic perspective view of an exemplary sterilizer system according to an aspect of the present invention;

FIG. 2a shows a schematic perspective view of an exemplary sterilizer system according to an aspect of the present invention;

FIG. 2b shows a schematic perspective view of an exemplary sterilizer system according to an aspect of the present invention;

FIG. 3 shows a schematic view of an exemplary steam sterilization apparatus of the exemplary sterilizer system according to an aspect of the present invention;

FIG. 4 shows a schematic view of an exemplary vaporized hydrogen peroxide sterilization apparatus of the exemplary sterilizer system according to an aspect of the present invention;

FIG. 5 shows a flow chart of an exemplary method of using an exemplary sterilizer for steam sterilization according to an aspect of the present invention;

FIG. 6 shows a flow chart of an exemplary method of using an exemplary sterilizer for sterilization with vaporized hydrogen peroxide according to an aspect of the present invention;

FIG. 7 shows a flow chart of an exemplary method of production of an exemplary sterilizer according to an aspect of the present invention;

FIG. 8 shows a flow chart of an exemplary method of production of an exemplary dosing channel of an exemplary sterilizer according to an aspect of the present invention; and

FIG. 9 shows a flow chart of an exemplary method of converting a steam autoclave to a hybrid sterilizer for steam sterilization and sterilization with vaporized hydrogen peroxide according to an aspect of the present invention.

### DETAILED DESCRIPTION

In one aspect the present invention is of a sterilizer. The sterilizer is configured to provide sterilization of at least one item with steam and to also provide sterilization of at least one item with vaporized hydrogen peroxide. In a further aspect, the present invention provides a method of using the sterilizer of the present invention to sterilize an item with steam. In a still further aspect, the present invention provides a method of using the sterilizer of the present invention to sterilize an item with vaporized hydrogen peroxide. The present invention also provides a process for converting a steam autoclave to a hybrid sterilizer providing steam sterilization and sterilization with vaporized hydrogen peroxide.

The sterilizer and methods of use thereof of the present invention have many advantages. A single sterilizer device can be used for sterilizing items with heat and can also be used for sterilization of heat sensitive items using vaporized hydrogen peroxide. Employing an alternative method to heat sterilization may lead to less wear and tear in some pieces of equipment. It is environmentally favorable to be able to have one device to perform both functions, minimizing the materials necessary in production. The device of the present invention is cheaper for the user than buying two separate systems. In addition, the hybrid device is less bulky and takes up less bench space than two devices.

As used herein the term ‘sterilization chamber’ may include, but is not limited to a compartment or suitable cavity where sterilization may be carried out.

As used herein the term ‘sterilization device’ may include, but is not limited to a sterilizer, which features a sterilization chamber or any suitable cavity for placement of an item to be sterilized. The sterilizer may provide the sterilization chamber or suitable cavity with sterilization conditions, materials, and other means for facilitating a sterilization process and sterilization of items in the sterilization chamber.

As used herein the term ‘sterilization’ may include, but is not limited to elimination, killing, removal or deactivation of biological agents, such as, but not limited to microorganisms, pathogens, bacteria, viruses, fungi, spore forms and prions from a specified region. Sterilization may be performed using a sterilization process and may run for longer than is required to provide a sterility assurance level. The term may include a process providing a sterility assurance level of at least 10⁻⁶.

As used herein the term ‘disinfection’ may include, but is not limited to a process to destroy microorganisms on a specified region. The term includes a method, which is less effective than sterilization at killing microorganisms and may not kill all microorganisms on a specified area, such as resistant bacterial spores.

As used herein the term ‘sterilization cycle’ may include, but is not limited to at least one stage of a sterilization procedure. The sterilization cycle may include all the stages of the sterilization procedure or only at least one stage.

As used herein the term ‘steam sterilization cycle’ may include a plurality of stages of a steam sterilization process, such as air removal, heating, sterilization, steam removal and drying. The air removal stage may include removing the air by methods, such as, but not limited to gravity, positive steam pulses and negative steam pulses with vacuum. The heating stage of the steam sterilization may include applying steam or water that is converted into steam for heating the load to the sterilization temperature. The sterilization stage may feature maintaining the temperature for a period of time approved for providing sterilization by use of steam or heated water. Removal of steam may be achieved by an outlet valve. The drying stage may include at least one of evacuating the steam, providing air and a vacuum.

As used herein the term ‘channel’ may include, but is not limited to a passage or a structure, which provides a passage or pathway for a material, such as a gas, vapor or liquid to flow along. The term may include a channel made from a material resistant to the material it is carrying. The term may include a channel for a separated passage and/or transportation of a material. The term may include a structure for directing the flow of a material contained in the channel and for conveying the material from one place to another for delivery of that material.

As used herein the terms ‘bypass system’, ‘means for blocking or providing access’, ‘access control means’ and ‘blocking element’ may be used interchangeably. The terms may include at least one component, which is configured to be manipulated to either or both block access or provide access to at least one other part of the sterilizer system.

As used herein the terms ‘hybrid system’, ‘hybrid sterilizer’ and ‘hybrid device’ may include, but are not limited to a system which can provide sterilization with steam and sterilization with vaporized hydrogen peroxide. The term may include a single unit.

As used herein the terms ‘first’ and ‘second’ or ‘one’ and ‘second’ may be used in connection with the vacuum pumps in a multi pump system. The terms may be used to distinguish one pump from the other in a multi pump system. The pump configured for use with steam may be referred to as the first pump when the pump configured for use with vaporized hydrogen peroxide is referred to as the second pump. The pump configured for use with steam may be referred to as the second pump when the pump configured for use with vaporized hydrogen peroxide is referred to as the first pump.

As used herein the term ‘connection’ may include, but is not limited to direct and indirect attachment or suitable coupling.

As used herein the terms ‘a’ and ‘an’ may mean ‘one’ or ‘more than one’.

As used herein the terms ‘comprising’, ‘including’, ‘containing’, ‘featuring’, ‘having’ and any forms of the terms thereof are inclusive and open ended and do not exclude additional elements or method steps, which are not recited.

The principles and operation of a hybrid sterilizer, as well as methods of use thereof according to the present invention may be better understood with reference to the figures. The figures show non-limiting aspects of the present invention.

The present invention provides a sterilizer system. The sterilizer system may provide cycles for steam sterilization and cycles for sterilization with vaporized hydrogen peroxide (VHP). The sterilizer includes a sterilization chamber in which an item to be sterilized can be placed. The sterilizer includes apparatus for providing steam sterilization and apparatus for providing vaporized hydrogen peroxide sterilization. The apparatus for providing steam sterilization may include at least some of the same components used in the apparatus for providing sterilization with vaporized hydrogen peroxide. The apparatus for providing sterilization with vaporized hydrogen peroxide may include at least some of the components used in the apparatus for providing sterilization with steam.

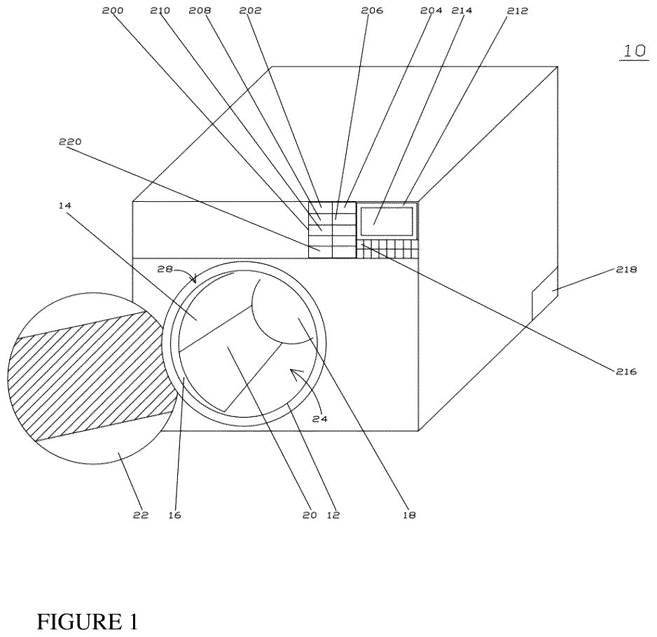

FIG. 1 shows a schematic view of an exemplary sterilizer system of the present invention 10. The sterilizer system includes a sterilization chamber 12. The sterilization system 10 may include one sterilization chamber 12, which can be used for sterilizing at least one item placed therein with steam and can also be used for sterilizing an item with vaporized hydrogen peroxide, depending on the selected type of sterilization. The sterilization chamber 12 may be a sterilization pressure vessel chamber. A pressure vessel chamber may feature a sealed chamber. The sealed chamber may be configured for maintaining a suitable vacuum in the chamber. The sterilization chamber 12 may be of any suitable size and shape. In some embodiments the chamber is in a cylindrical shape. In a cylindrical shaped chamber, the sterilization chamber 12 may be constructed of at least one side wall 14, a front wall 16 and a back wall 18 configured about a tubular shaped cavity 20 for accommodating items to be sterilized. The front wall 16 may be defined as the wall which includes a door 22 for providing a user with access to the interior 20 of the chamber. The back wall 18 may be the opposite facing wall to the front wall 16. The sterilization chamber 12 has an inner surface 24 and an external surface 28. The sterilization chamber 12 and the door 22 may be made from a material which is heat resistant and resistant to hydrogen peroxide. One non-limiting example of a suitable material is stainless steel. The sterilization chamber may be conducive for sterilizing any suitable item, such as but not limited to medical, surgical, cosmetic and veterinary instruments. The sterilization chamber may be configured for sterilization of at least one item with steam or vaporized hydrogen peroxide. The sterilization chamber may include supports for trays or shelves to hold items to be sterilized. The supports and trays may be constructed from materials resistant to heat and to hydrogen peroxide.

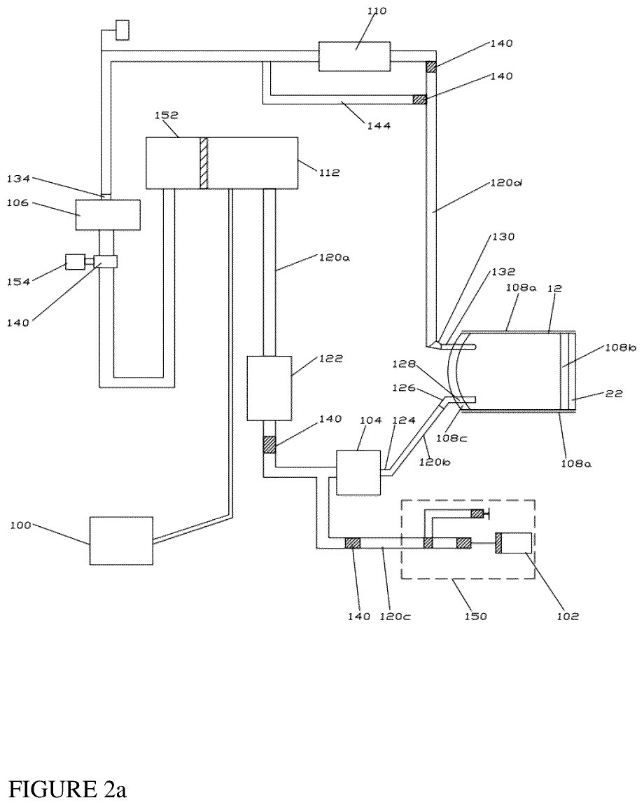

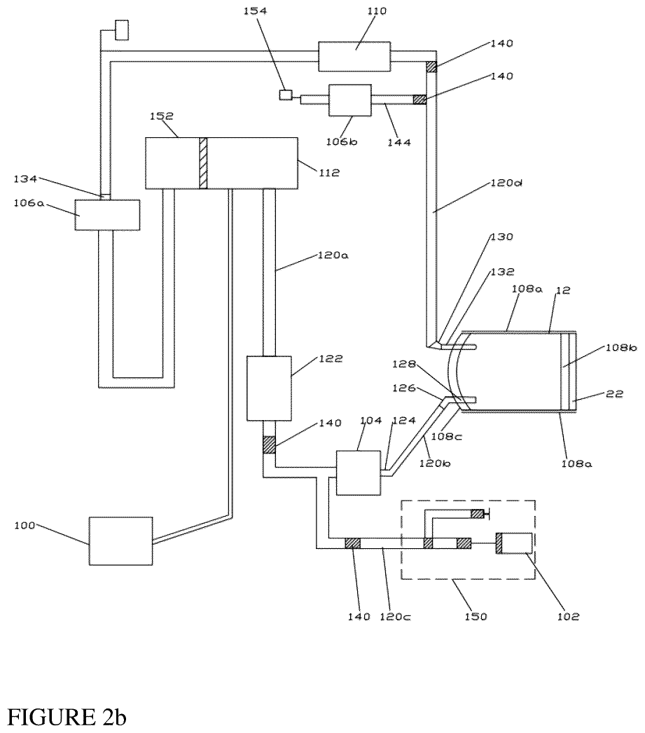

The sterilizer system features apparatus for providing either steam or vaporized hydrogen peroxide as shown schematically in FIG. 2a. The apparatus may include a water source 100, a source of hydrogen peroxide 102 and a vaporizer 104 for generating from the respective sources, steam and vaporized hydrogen peroxide. As shown in FIG. 2a, the apparatus may include one vaporizer 104, which is configured for generating both steam and vaporized hydrogen peroxide from water and liquid hydrogen peroxide respectively. In some embodiments, the apparatus may include a plurality of vaporizers 104 (not shown in FIG. 2a). In one non-limiting example the apparatus may include two vaporizers 104, wherein one vaporizer 104 is configured to convert water to steam and the other vaporizer 104 is configured to convert liquid hydrogen peroxide to vaporized hydrogen peroxide. The apparatus may include a pump 106 for evacuating air from the sterilization chamber 12. As shown in FIG. 2a, the apparatus may include one pump 106, which is configured to be used in both a steam sterilization cycle and a vaporized hydrogen peroxide sterilization cycle. In some embodiments as shown schematically in FIG. 2b, the apparatus may include a plurality of pumps 106a, 106b, where each pump is configured to be used with a different sterilization medium. In one non-limiting example as shown in FIG. 2b the apparatus may include two pumps 106, wherein one pump 106a is configured to be used in a steam sterilization cycle and the second pump 106b is configured to be used in a vaporized hydrogen peroxide sterilization cycle. The apparatus may include at least one heater 108 for heating the chamber 12 and contents contained therein. The apparatus may include a cooler 110 for cooling hot air and steam expelled from the sterilization chamber 12. The cooler 110 may be used only in steam sterilization. The system may feature delivery channels 120 configured for delivering and conveying at least one material, such as, but not limited to water, steam, liquid hydrogen peroxide, vaporized hydrogen peroxide, air, byproducts thereof and mixtures thereof. Non limiting examples of suitable delivery channels 120 include pipes and tubes. In some embodiments, one delivery channel may feature a plurality of lengths of tubing in order to provide a channel through components of the system, such as pumps and vaporizers. In such an embodiment one length of the tubing may be connected at a distal end to an inlet of a component and a second length of tubing may be connected at a proximal end to the outlet of the same component. The system may include at least one delivery channel 120a for transporting water from a water storage container 112 to the vaporizer 104. The water delivery channel 120a may include a pump 122, such as, but not limited to a water pump to propel the water towards and through the vaporizer 104. The delivery channels 120 may include at least one channel 120b, which may be connected at one end 124 to the vaporizer and at a second end 126 to an inlet 128 of the sterilization chamber 12 for transporting steam or vaporized hydrogen peroxide into the sterilization chamber 12. The delivery channels 120 may include a channel 120c for transporting liquid hydrogen peroxide from a source of liquid hydrogen peroxide 102 to the vaporizer 104. The delivery channels 120 may include at least one channel 120d for transporting expelled air and other material pumped out of the sterilization chamber 12. The channel for expelled material 120d may be connected at one end 130 to an outlet 132 of the sterilization chamber 12 and at a second end 134 to the vacuum pump 106. The sterilizer system may include a plurality of means 140, such as access control means which can be manipulated to reversibly provide access or block a delivery channel 120. The sterilizer system may also include at least one bypass channel 144 to bypass a component, such as, but not limited to the cooler 110. The sterilizer system may include a dosing system 150 for providing a volume of liquid hydrogen peroxide to the vaporizer 104 to be vaporized by the vaporizer. The system may feature at least one waste reservoir 152, such as a storage container 152 for storing wastewater produced from the expelled air and steam. The system may include at least one component 154 for trapping and storing waste hydrogen peroxide and byproducts produced from and contained in the material expelled by the pump 106.

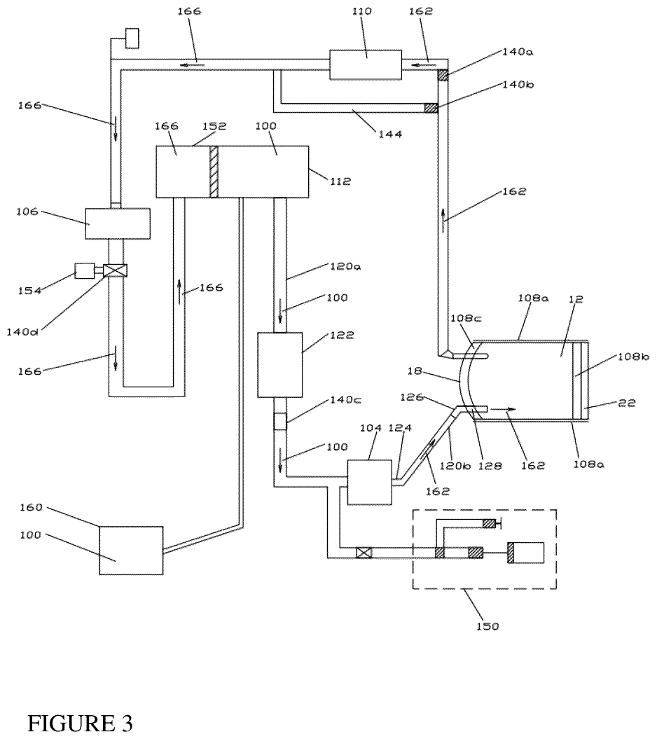

The apparatus of the hybrid sterilizer system for providing steam sterilization to the chamber is shown in more detail in FIG. 3. The steam sterilization apparatus may include a source of steam 100. The source of steam 100 may be water. Water 100 may be stored in a water storage device 112, such as, but not limited to a tank 112. In some embodiments, water 100 may be supplied to the water storage device 112 from a water inlet 160. In some embodiments, the water inlet 160 may be operated automatically. The steam sterilization apparatus may also include a waste storage device 152 for storing wastewater 166, such as but not limited to condensed steam expelled from the chamber 12.

The water 100 may be conveyed and suitably distributed through at least one suitable channel 120a configured to hold the water 100 and direct its flow to the relevant areas in the system. Non limiting examples of water delivery channels 120a include tubing and pipes. The tubing 120a may be made from a water and heat resistant material such as, for example silicone. The flow of the water 100 in the at least one delivery channel 120a may be facilitated by a pump 122, such as a water pump. The water 100 may be propelled through the water delivery channel 120a by the water pump 122 to a vaporizer 104. The vaporizer 104 may be a heat exchanger, which is configured to convert the water 100 to steam 162. The vaporizer 104 may be configured so that it is also suitable for vaporizing liquid hydrogen peroxide to vaporized hydrogen peroxide. The vaporizer 104 may be made from a material resistant to hydrogen peroxide and heat and approved for medical use. One non-limiting example of a suitable vaporizer 104 is an anodized coated aluminum heat exchanger.

The steam 162 produced by the vaporizer 104 may be carried by a delivery channel 120b into the sterilization chamber. In some embodiments, the steam delivery channel 120b is a Teflon tube, which may be connected at a first end 124 to the vaporizer 104 and at a second end 126 to an inlet 128 of the sterilization chamber 12. The steam 162 may be supplied to the chamber 12 via the at least one inlet 128 in the chamber 12. The chamber may include a plurality of inlets and outlets. In one embodiment the chamber may have about four inlets and outlets. One inlet may be configured to inlet either steam or vaporized hydrogen peroxide depending on the sterilization medium used. One outlet may be configured as an exhaust for steam or vaporized hydrogen depending on the sterilization medium used. The chamber may include inlets and outlets configured for air removal, for inserting air, as a safety pressure release valve and for a pressure measurement element. At least one item, which has been placed in the chamber 12 for sterilization may be exposed to the steam 162 for sterilization of the at least one item.

The sterilization chamber 12 may be heated by at least one heater 108. In some embodiments the sterilization chamber 12 may include a heater 108a which surrounds the external surface of the chamber, such as a jacket heater 108a. In some embodiments, the system may include additional heaters 108. The additional heaters 108 may include at least one of a heater 108b attached to the door 22 of the sterilization chamber 12 and a heater 108c attached to the opposite back wall 18 of the sterilization chamber 12. In some embodiments any number of the heaters 108 may be turned on to provide heat during the sterilization process. In some embodiments of a steam sterilization cycle only the surrounding jacket heater 108a is used to heat the sterilization chamber 12. The sterilization chamber may be heated to any suitable temperature. The temperature may be dependent on the sterilization cycle used. In some embodiments of the sterilization stage in the steam cycle, the sterilization chamber 12 may be heated to a temperature of from about 110° C. to about 150° C. In one exemplary steam cycle the sterilization chamber 12 may be heated to a temperature of from about 121° C. to about 124° C. In another exemplary steam cycle the sterilization chamber 12 may be heated to a temperature of from about 132° C. to about 135. C. In an additional exemplary steam cycle the sterilization chamber 12 may be heated to a temperature of from about 134° C. to about 137° C. In some embodiments, in the drying stage of the steam sterilization cycle, the same heater/s 108 may be used to dry the at least one item that has been sterilized.

As stated above, the sterilizer system includes a pump 106 to expel the air from the sterilization chamber 12. The pump 106 may be a vacuum pump. One non-limiting example of a suitable vacuum pump 106 is a diaphragm vacuum pump. The components of the pump 106 may be configured to work with both steam and vaporized hydrogen peroxide. In some embodiments, the hot air and steam 162 which is expelled by the pump 106 needs to be cooled. Cooling may prevent overheating of the pump 106. The system includes a cooler 110 for this purpose. The cooler 110 may be a heat exchanger configured for cooling. A suitable cooler 110 may be made from brass. Brass is not compatible with hydrogen peroxide and as such, the system is configured to protect a brass containing cooler 110 from exposure to hydrogen peroxide. Other non-limiting examples of suitable materials from which the heat exchanger 110 may be constructed include aluminum and copper.

As described above the sterilizer system may include a plurality of access control means 140, for blocking or providing access to different parts of the sterilizer system depending on the type of sterilization medium used. The plurality of access control means 140 can be manipulated to provide access or block a delivery channel 120. A suitable access control means 140 may include a valve 140 configured to reversibly block or open a channel 120. The valve 140 may be any suitable valve, which may be a multi-way valve. The plurality of access control means 140 may be part of a bypass system, which by blocking access to a component can bypass that component.

An access control means, such as a valve or tap 140a may be attached proximal to the inlet of the cooler 110. The valve 140a or tap may be open or suitably configured as shown in FIG. 3 when hot air 162 is being sucked out of the chamber 12 during steam sterilization, to provide the hot air and steam 162 with access to the cooler 110. Cooling the hot air and steam 162 may convert it into a liquid, such as wastewater 166. The valve or tap 140a may be closed or suitably configured during sterilization with vaporized hydrogen peroxide, in order to prevent the brass containing cooler 110 from being exposed to hydrogen peroxide. A valve or tap 140b may be attached adjacent to the proximal end of a bypass channel 144, the bypass channel for bypassing the cooler 110. In a steam sterilization cycle, the valve or tap 140b may be closed or suitably configured in order to block access to the bypass channel 144 and ensure all the expelled hot air and steam 162 are pumped through the cooler 110 to be cooled by the cooler 110.

An access control means, such as a valve or tap 140c may be disposed between the distal end of the water pump 122 and the proximal end of the vaporizer 104. The valve or tap 140c may be open during relevant stages of the steam sterilization cycle to provide the water with an unblocked path to the vaporizer 104 and allow the water to be pumped to the vaporizer 104. The valve or tap 140c may be closed during a vaporized hydrogen peroxide cycle.

In an embodiment wherein there is a trap 154 applied adjacent to an outlet of the pump 106, the trap may filter any residue of hydrogen peroxide. The trap 154 may facilitate eliminating the odor of the hydrogen peroxide. An access control means such as a valve or tap 140d may be disposed about the pump 106 exit outlet proximal to the trap 154. The access control means such as a valve 140d may be manipulated depending on the medium being used in the sterilization cycle to either facilitate filtering of hydrogen peroxide residue, or facilitate providing a path in the delivery channel for delivering extracted water to the wastewater tank 152. The valve 140d may be a three-way valve and may be operated to prevent any hydrogen peroxide or other byproducts from affecting components of the steam sterilization apparatus during a steam sterilization cycle. The valve 140d may be configured during a steam sterilization cycle to block access to the trap 154 and prevent any hydrogen peroxide residue from entering the wastewater storage container. The valve 140d may be manipulated so that the cooled expelled air and water and other byproducts 166, which are pumped out of the chamber 12 are provided with access to a delivery channel into the wastewater storage container 152 for pumping of the wastewater 166 into the wastewater storage container 152.

When a steam sterilization cycle is selected, the apparatus for providing steam sterilization is used according to the herein description. The system may be reconfigured by the opening of the valves relating to the steam channel pathway, such as, but not limited to valves 140a, 140c, 140d to provide access to these channels and the closing of valves 140h and 140b respectively relating to the hydrogen peroxide dosing channel and bypass channel to reversibly block access to these channels. The chamber 12 may be heated and the vacuum pump 106 may extract air from the chamber 12. Water may be propelled by the water pump 122 to the vaporizer 104 for conversion to steam. The steam may be introduced into the heated chamber 12. Hot air extracted by the vacuum pump 106 may be cooled by the cooler 110 to result in water which may be directed to a wastewater tank 152. The steam sterilization apparatus and the production of the steam and its supply to the sterilization chamber using the described apparatus may be continued until successful sterilization of the at least one item being sterilized.

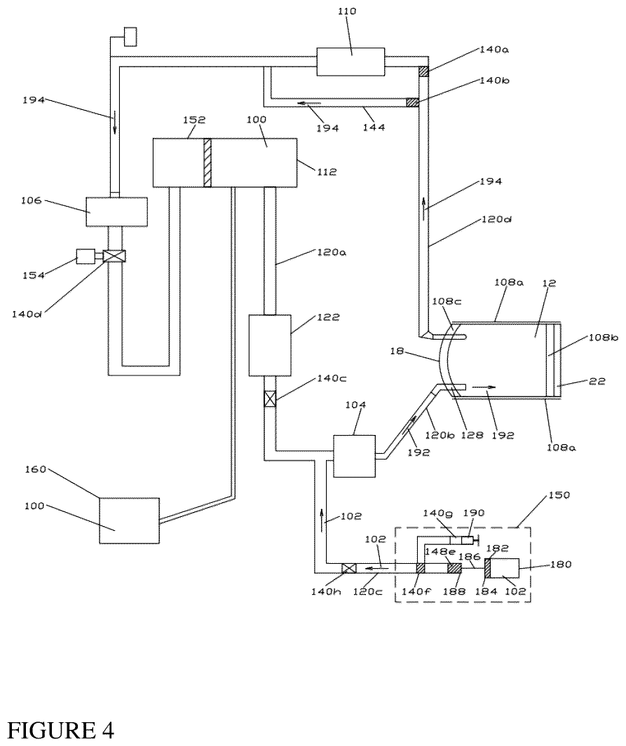

The apparatus of the sterilizer system for providing vaporized hydrogen peroxide sterilization to the sterilization chamber is shown in more detail in FIG. 4. FIG. 4 shows a schematic view of an exemplary system for providing sterilization with vaporized hydrogen peroxide. The vaporized hydrogen peroxide sterilization system may include a source of vaporized hydrogen peroxide 102. The source of vaporized hydrogen peroxide may be liquid hydrogen peroxide 102. The source of vaporized hydrogen peroxide may be any suitable source. In some embodiments the suitable source of vaporized hydrogen peroxide may be in a suitable state for conversion into vaporized hydrogen peroxide. The source of vaporized hydrogen peroxide may be converted to vaporized hydrogen peroxide by the system of the present invention. Any suitable concentration of liquid hydrogen peroxide 102 may be used. In some embodiments a concentration of from about 45 percent to about 50 percent of liquid hydrogen peroxide is used. The liquid hydrogen peroxide may be stored in a container 180. In some embodiments the liquid hydrogen peroxide 102 is kept in a bottle 180. In some embodiments, one bottle may be sufficient for one vaporized hydrogen peroxide sterilization cycle. In some embodiments, one bottle is sufficient for multiple vaporized hydrogen peroxide sterilization cycles. The bottle 180 may have a top 182 configured for extraction of the liquid hydrogen peroxide 102. In one non-limiting example, the top 182 may be fitted with a septum cap 184. Extraction of the liquid may be done with a needle 186, such as a syringe type needle with a bore for accommodating a liquid.

As stated above, the vaporized hydrogen peroxide sterilization system may feature a dosing system 150 for providing a volume of liquid hydrogen peroxide 102 to the vaporizer 104 to be vaporized into vaporized hydrogen peroxide. The dosing system 150 may be any suitable apparatus for providing a volume of liquid hydrogen peroxide 102 to the vaporizer 104. The dosing system 150 may include the following elements, such as but not limited to a needle 186, a delivery channel 120c and a plurality of valves 140. The needle 186 with a central bore, may be connected to an inlet 188 configured for administering liquid hydrogen peroxide 102 to a delivery channel 120c, which is configured to hold and deliver liquid hydrogen peroxide to the vaporizer 104. The needle 186 may be fixedly attached to the system. The needle 186 may be fixedly attached to the proximal extremity of the delivery channel 120c at an inlet. The needle 186 is made from a material resistant to hydrogen peroxide, such as but not limited to stainless steel. A sufficient amount of liquid hydrogen peroxide 102 may be withdrawn using the needle 186. The amount may depend on the load, the type of cycle and other parameters of the cycle. In some embodiments the amount of liquid hydrogen peroxide withdrawn may be from about 1 ml to about 10 ml. In some embodiments the amount of liquid hydrogen peroxide withdrawn may be from about 3 ml to about 6 ml. The needle 186 may be suitably coupled to the system 150 so that it can pierce the top 182 of a bottle 180 of liquid hydrogen peroxide 102. The liquid hydrogen peroxide 102 can be drawn from the bottle via the needle 186 into the hydrogen peroxide inlet delivery channel 120c. The piercing of the bottle 180 and the introduction of the liquid hydrogen peroxide 102 into the delivery channel 120c can be done manually or can be done automatically. In some embodiments the inlet delivery channel 120c can include a blocking switch (not shown) to prevent additional hydrogen peroxide from entering the channel 120c. In some embodiments the bottle 180 of liquid hydrogen peroxide may have a metered mechanism for expelling a specific volume of liquid each time it is activated and then sealing the bottle until the next activation. In one non-limiting example a bottle 180 may contain the amount of liquid hydrogen peroxide needed for a single vaporized hydrogen peroxide sterilization cycle.

The dosing mechanism 150 may feature a plurality of valves 140, which are connected to the inlet delivery channel 120c. In one embodiment, there are two spaced apart valves 140e, 140f disposed about a small section of the delivery channel 120c in proximity and distal to the inlet 188 for the liquid hydrogen peroxide 102. The valves 140e, 140f may be manipulated such as by opening and closing to provide the same low pressure as found in the sterilization chamber 12 to the section of the channel 120c situated between the valves 140e, 140f. In this way, the opening and closing of the valves 140e, 140f supply the volume of liquid hydrogen peroxide 102 expelled from the bottle 180 into the inlet delivery channel 120c. The dosing mechanism 150 may also include an air inlet 190 which can be closed and opened by an air inlet valve 140g. The air inlet valve 140g may be opened to allow air at atmospheric pressure to enter the inlet delivery channel 120c to propel the volume of liquid hydrogen peroxide 102 to the vaporizer 104 and through the vaporizer 104.

In one non-limiting example of the system for providing vaporized hydrogen peroxide sterilization to the sterilization chamber the system may include an alternative dosing mechanism 150 (not shown in the figures) featuring a dosing pump. The dosing pump may extract a volume of liquid hydrogen peroxide from a storage container such as a vial. The extracted dose may be introduced to the vaporizer by any suitable means, such as using a syringe and appropriate tubing attached to the vaporizer.

A further non-limiting example of an alternative dosing mechanism 150 (not shown in the figures) may include manual administration of a volume of liquid hydrogen peroxide, which is extracted from a storage container such as a vial and injected into the system in a suitable way so that it enters the vaporizer for conversion into vaporized hydrogen peroxide.

The vaporizer 104 used by the vaporized hydrogen peroxide sterilization system may be the same vaporizer as described hereinabove for the steam sterilization apparatus. As stated, the vaporizer 104 is constructed from materials which are compatible with both hydrogen peroxide, water and heat. As such, the sterilization system may only need one vaporizer 104, which can be used in either a steam sterilization cycle or a vaporized hydrogen peroxide sterilization cycle.

The liquid hydrogen peroxide 102 is vaporized by the vaporizer 104 and the resulting vaporized hydrogen peroxide 192 may be carried and conveyed by a vaporized hydrogen peroxide delivery channel 120b into the sterilization chamber 12. In some embodiments, the vaporized hydrogen peroxide delivery channel 120b is a Teflon tube. This may be the same tube 120b as the steam delivery channel 120b configured to connect to the inlet 128 of the sterilization chamber 12 and deliver steam as previously described. The vaporized hydrogen peroxide 192 may enter the sterilization chamber 12 via the at least one inlet 128 of the chamber 12 to which the vaporized hydrogen peroxide delivery channel 120b is connected. The at least one inlet 128 may be the same inlet 128 used to provide steam to the chamber 12 during a steam sterilization cycle. In some embodiments the at least one inlet 128 for providing vaporized hydrogen peroxide to the chamber 12 is one inlet. The at least one item placed in the chamber 12 for sterilization may be exposed to the vaporized hydrogen peroxide 192 for sterilization of the at least one item with the vaporized hydrogen peroxide 192.

The vaporized hydrogen peroxide sterilization apparatus includes a pump 106, such as a vacuum pump 106 to expel the air from the sterilization chamber 12 as previously described. The same pump 106 is used in either a steam sterilization cycle or a vaporized hydrogen peroxide sterilization cycle. In vaporized hydrogen peroxide sterilization, the expelled air 194 does not need to be cooled. The expelled air may be expelled via a delivery channel 120d configured for holding and transporting the expelled material. The expelled waste products may be filtered by a trap 154, such as a filter 154, which may be disposed adjacent to the distal end of the outlet of the pump 106. The filter 154 may absorb any hydrogen peroxide and other waste products. One non-limiting example of a suitable trap 154 includes an active carbon filter.

The sterilization chamber 12 may be heated by at least one heater 108. In some embodiments the sterilization chamber 12 may include at least one heater 108a which surrounds the external surface of the chamber (periphery heater), at least one heater 108b attached to the door 22 of the sterilization chamber 12 and at least one heater 108c attached to the opposite back wall of the sterilization chamber as previously described. In some embodiments any suitable number of the heaters 108 may be activated during, prior and after the vaporized hydrogen peroxide sterilization process. In some embodiments during a vaporized hydrogen peroxide sterilization cycle the surrounding heater 108a and back 108c and front heaters 108b are all used. The heating may facilitate avoiding, or reducing hydrogen peroxide condensation. In some embodiments when the vaporized hydrogen peroxide contacts surfaces which have not been warmed, the vaporized hydrogen peroxide will not remain in a vaporized state. The heating may facilitate raising the temperature of the items placed in the sterilization chamber to a sufficient temperature in order to prevent the vaporized hydrogen peroxide from liquefying. In some embodiments the heaters are configured to heat the items to at least 50° C. The temperature used may depend on factors such as the concentration of hydrogen peroxide and the vacuum. In embodiments wherein the chamber has a higher vacuum and/or lower hydrogen peroxide concentration the items can be heated to a lower temperature for a similar effect. The sterilization chamber 12 may be heated to a relatively cool temperature of from about 50° C. to about 60° C. In some embodiments the sterilization chamber may be heated to a temperature of from about 55° C. to about 57° C. The temperature may be a temperature, which is safe for a heat resistant material. The same heating devices may be used in the drying stage of the sterilization cycle after sterilization has been achieved.

As stated above the sterilizer system may include a plurality of access control means 140, for reversibly blocking or providing access to different parts of the sterilizer system depending on the type of sterilization medium used. The plurality of access control means 140, such as valves, may be configured for providing access to hydrogen peroxide delivery channels, for protecting devices which are sensitive to hydrogen peroxide and for reversibly blocking water delivery channels associated with a steam sterilization cycle. The valves 140 may be one-way valves or multi-way valves.

An access control means, such as a valve or tap 140a may be attached proximal to the inlet of the cooler. The valve or tap 140a may be closed or suitably reversibly configured when air is being sucked out of the chamber 12 during vaporized hydrogen peroxide sterilization, to prevent access to the cooler 110 of the expelled air, in order to prevent the brass containing cooler from being exposed to hydrogen peroxide. The expelled air in a vaporized hydrogen peroxide cycle does not need to be cooled by the cooler. A bypass channel 144 for bypassing the cooler 110 may be included, the bypass channel 144 for expelling air from the sterilization chamber 12 during a vaporized hydrogen peroxide sterilization cycle. A valve or tap 140b may be attached adjacent to a proximal end of the bypass channel 144. In a vaporized hydrogen peroxide sterilization cycle, the valve or tap 140b may be opened or suitably reversibly configured in order to circumvent the cooler 110 and provide the expelled air with access to the bypass channel 144.

At least one access control means 140 may be employed to block water and steam delivery channels during a vaporized hydrogen peroxide sterilization cycle At least one access control means 140 may be for reversibly blocking off at least part of the steam sterilization apparatus. An access control means 140, such as a valve or tap 140c may be disposed between the distal end of the water pump 122 and the proximal end of the vaporizer 104. The valve or tap 140c may be open or suitably configured during the steam sterilization cycle to allow water to be pumped to the vaporizer 104. The valve or tap 140c may be closed or suitably configured during a vaporized hydrogen peroxide cycle to block water in the water delivery channel 120a from entering the vaporizer 104 or any hydrogen peroxide delivery channel.

At least one valve 140h may be attached to the liquid hydrogen peroxide delivery channel 120c in a suitable position to control access of hydrogen peroxide to the vaporizer 104 and sterilization system. In some embodiments wherein the vaporized hydrogen peroxide sterilization apparatus includes dosing valves, such as 140e, 140f, the at least one valve 140h may be disposed in a position spaced apart and distal from the two dosing valves 140e, 140f. When the valve 140h is closed, it blocks the liquid hydrogen peroxide delivery channel from the sterilization system, as is the system setting recommended during a steam sterilization cycle. In the vaporized hydrogen peroxide sterilization cycle, the valve 140h is open to provide liquid hydrogen peroxide in the liquid hydrogen peroxide delivery channel 120c with access to the vaporizer 104 and sterilization system.

In an embodiment wherein there is a filter 154 applied adjacent to the distal end of an outlet of the pump 106, an access control means such as a valve or tap 140d may be disposed about the pump exit outlet proximal to the filter 154. The valve 140d may be a multi-way valve, such as a three-way valve. The valve 140d may be appropriately manipulated in order to block any residue of hydrogen peroxide from affecting components of the steam sterilization apparatus during a steam sterilization cycle. During the vaporized hydrogen peroxide sterilization cycle the valve 140d may be open to open the path to the filter 154 for access to it by the residue of hydrogen peroxide, so that the residue can be filtered by the filter 154.

When a sterilization cycle using vaporized hydrogen peroxide is selected, the apparatus for providing vaporized hydrogen peroxide sterilization may be used according to the herein description. The system may be reconfigured for providing this cycle by the reversible blocking of access to and from the water delivery channel 120a, such as with valve 140c, and the reversible blocking of access to the cooler 110, such as with valve 140a. The system may be reconfigured by the providing of access to the liquid hydrogen peroxide delivery channel, such as with valve 140h, the opening of a bypass channel 144 for bypassing the cooler 110, such as with valve 140b and the opening of access to the filter 154, such as with valve 140d and the resulting closing of access to the wastewater tank 152. The chamber 12 may be warmed and the vacuum pump 106 may extract air from the chamber 12. A volume of liquid hydrogen peroxide may be provided to the vaporizer 104 as previously described in more detail, for conversion to vaporized hydrogen peroxide. The vaporized hydrogen peroxide may be introduced into the warmed chamber 12. Air extracted by the vacuum pump 106 may be directed via the bypass channel 144 to bypass the cooler 110 and the extracted air which may contain hydrogen peroxide and products thereof may be directed to the filter 154. The vaporized hydrogen peroxide sterilization apparatus and the production of the vaporized hydrogen peroxide and its supply to the sterilization chamber 12 using the described apparatus may be continued until successful sterilization of the at least one item being sterilized.

The vaporized hydrogen peroxide sterilization apparatus is configured for drying of the sterilized items. The apparatus may be configured to provide pulses of air and vacuum for drying the item. The apparatus may include any components which may employ any suitable method for drying the sterilized items.

As disclosed hereinabove, in some embodiments the sterilization system may include a plurality of pumps 106, such as shown schematically in FIG. 2b, which is now described in more detail. The arrangement and structure of such a system may differ from a sterilization system, which employs one vacuum pump 106 to be used during either the steam sterilization or vaporized hydrogen peroxide sterilization cycles, as shown in FIG. 2a. The apparatus and components of the multi pump system may be the same as for the one vacuum pump system, except for the addition of a vacuum pump 106b and the different position of the additional vacuum pump 106b and the filter 154 in the sterilization system. The apparatus and components in the multi pump system, which do not differ from the one pump system will not be described again to avoid repetition and are as previously described. In the system with a plurality of vacuum pumps, the bypass channel 144, which is configured to bypass the cooler 110 is not reconnected to the delivery channel 120 at a position distal to the cooler 110 as was described in the system shown in FIG. 2a. In the multi pump system, an additional pump, such as a vacuum pump 106b may be connected at a suitable position about the bypass channel 144. The additional pump 106b is configured for being used only in a sterilization cycle employing hydrogen peroxide and may pump out air and vaporized hydrogen peroxide sterilization and byproducts from the sterilization chamber through the distribution channel 120d and into the bypass channel 144. A non-limiting example of a suitable pump includes an oil sealed vacuum pump. A filter 154 as previously described, may be coupled in any suitable way to the distal extremity of the pump 106b in order to trap and store waste hydrogen peroxide and byproducts produced from and contained in the material expelled by the pump 106b during sterilization with vaporized hydrogen peroxide. In some embodiments, the filter 154 may be disposed at an extremity of the vaporized hydrogen peroxide apparatus of the system. The disconnection of the bypass channel 144 from the distribution channels distal to the cooler 110 and the position of the vaporized hydrogen peroxide vacuum pump 106b and trap 154 about the bypass channel prevents the steam vacuum pump 106a and waste water channels and storage containers from being exposed to hydrogen peroxide and its byproducts.

Referring back to FIG. 1, the sterilization system may include a control 200. The control 200 may include a switch 202 to start and stop the sterilization device. The control may include power controlling means 204. The control 200 may include different programs of sterilization cycle 206. The control 200 may control which type of sterilization cycle is employed to sterilize at least one item 208. In some embodiments the control 200 may choose the optimal type of sterilization cycle depending on the type of item being sterilized. A user may select whether to perform sterilization with steam or with vaporized hydrogen peroxide 210. The system control 200 may automatically identify an item, or a user may convey to the control what type of item is being sterilized. Information relating to the item material may be stored or uploaded. When a cycle is selected, the control 200 may automatically open and close access to the delivery channels and components of the sterilization apparatus according to whether steam or vaporized hydrogen peroxide is being used. The control 200 may include a means for opening and closing inlets and outlets. The control 200 may include any suitable electronics, a computer and software. The control 200 may include a screen 212, a display 214 and a keyboard 216.

The sterilization system 10 may include a power supply 218. The sterilization system may be mains operated. The power supply may provide the power for activation of the system and the sterilization cycles. Suitable power sources that may provide sufficient power may include at least one of AC, DC, battery, photovoltaic power source, solar power source, RF and microwave. In some embodiments, the power source is an AC supply. In some embodiments, the power source is a generator. The power source may provide power in the range of from about 1000 W to about 2000 W. In some embodiments, the power source may provide at least 1500 W.

The sterilization system 10 may include a timer 220. The timer 220 may be for setting the time duration of the sterilization cycle. The timer 220 may be used to stop the sterilization cycle after a predetermined time or requisite time when sterilization and drying are complete. The sterilization system 10 may include an indicator to indicate when sterilization is achieved. The sterilization system 10 may include at least one sensor. Sensors may include, but are not limited to temperature sensors, pressure sensors, light sensors, water detection sensors, hydrogen peroxide detection sensors and bacteria detection sensors.

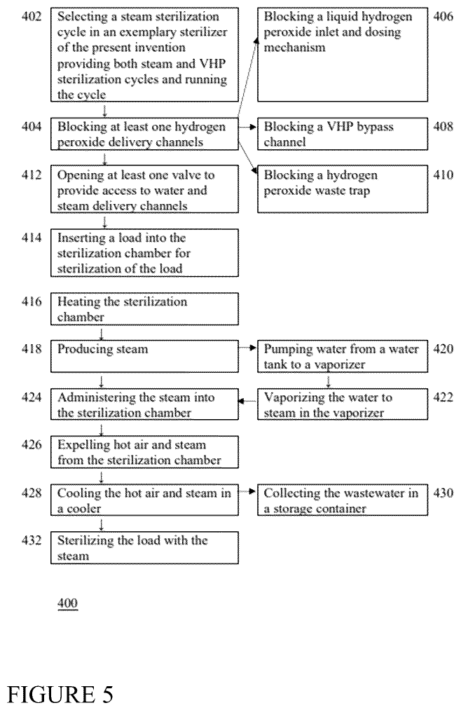

The present invention provides methods of using the sterilization system of the present invention The present invention provides a method of sterilizing an item using steam sterilization in a hybrid sterilization system of the present invention 400 as shown schematically in FIG. 5. The order of the steps is not meant to be limiting and any suitable order may be used.

A sterilization cycle may be selected and run. A user may select a steam sterilization cycle 402. In one embodiment, the computer may determine the optimal sterilization cycle for the item being sterilized, which may be a steam sterilization cycle.

When the type of sterilization medium has been chosen as steam, the control of the system may block any of the hydrogen peroxide delivery channels, which may interfere with the steam sterilization cycle or be affected by the steam sterilization cycle 404. A liquid hydrogen peroxide inlet delivery channel and dosing mechanism may be blocked with any suitable access control means configured for blocking such as, but not limited to a valve, which can close the channel until needed 406. A vaporized hydrogen peroxide bypass channel for bypassing a cooler may be blocked by a suitable access control means, which is configured for reversible blocking of access such as, but not limited to a valve or tap 408. A trap, such as a filter for absorbing hydrogen peroxide waste may be blocked 410. The filter may be adjacent and distal to the outlet of a vacuum pump. In an embodiment wherein the system includes a plurality of vacuum pumps, wherein each vacuum pump is configured to be used for either only steam sterilization or only vaporized hydrogen peroxide sterilization, access to the vacuum pump configured for vaporized hydrogen peroxide sterilization is blocked. In such an embodiment, the vacuum pump configured for vaporized hydrogen peroxide sterilization is coupled to the bypass channel so that blocking the bypass channel 408 also blocks access to the vaporized hydrogen peroxide pump. The outlet of the vacuum pump configured for vaporized hydrogen peroxide sterilization is coupled to the trap configured for absorbing hydrogen peroxide and its byproducts and therefore blocking of the bypass channel 408 also blocks access to the trap. The trap may not need to be separately blocked as in the one vacuum pump system.

When the type of sterilization medium has been chosen as steam, the control of the system may open any of the water and steam delivery channels, which are used in the steam sterilization cycle 412. The delivery channels may feature devices to block or provide access to the channels, such as suitable valves or taps. A valve may be opened to provide the pumped water with access to the vaporizer. A valve may be opened to provide the extracted steam and hot air with access to the cooler.

A load, such as at least one item may be inserted in any suitable way in the sterilization chamber 414. The at least one item may be placed on a shelf. The at least one item may be wrapped or unwrapped. In some embodiments the chamber and load may be heated pre sterilization 416. The preheating may be achieved using pulses of steam and vacuum. In one embodiment, the sterilization chamber may be heated in a pre-sterilization heating stage additionally or alternatively by a heating source, such as a heating jacket disposed about the periphery of the sterilization chamber. In some embodiments additional heaters are used, such as a door heater and a back wall heater. The pre sterilization heating stage may be for any suitable length of time. Pre sterilization heating may include heating to any temperature below the sterilization temperature. Pre sterilization heating may reduce the amount of condensation in the sterilization chamber and on the item being sterilized. After the chamber is preheated, the at least one heater may be used to heat the chamber and load during sterilization. In the sterilization stage the chamber may be heated to any suitable temperature. The temperature may be dependent on the sterilization cycle used. In some embodiments of the sterilization stage in the steam cycle, the sterilization chamber 12 may be heated to a temperature of from about 110° C. to about 150° C. In one exemplary steam cycle the sterilization chamber 12 may be heated to a temperature of from about 121° C. to about 124° C. In another exemplary steam cycle the sterilization chamber may be heated to a temperature of from about 132° C. to about 135° C. In an additional exemplary steam cycle the sterilization chamber may be heated to a temperature of from about 134° C. to about 137° C.

Steam may be produced 418. In some embodiments, steam may be produced from water stored or supplied to the system. Water may be stored in a water storage device, such as a tank. The water tank may be supplied with water from a water inlet. In some embodiments, the water supply may be controlled by the controller. In some embodiments a user may be notified if the water tank needs refilling. A pump, such as a water pump may propel the water through water delivery channels. The water delivery channels may be tubing. The water may be transported to a vaporizer 420. In some embodiments, the system may include an access control means for blocking the water delivery channel, which may be positioned between the distal end of the water pump and the proximal end of the vaporizer. The control can open and close the access control means according to need. The water may be vaporized by the vaporizer 422 and the resulting steam may be administered into the sterilization chamber via an inlet in the sterilization chamber 424.

A valve adjacent to the outlet of a pump such as a vacuum pump may be reconfigured so that access to a waste hydrogen peroxide filter is blocked and access to a delivery channel to the waste water tank is open. In an embodiment wherein the system includes a plurality of vacuum pumps, and where two separate vacuum pumps are used, one for steam sterilization and one for vaporized hydrogen peroxide sterilization, there is no need to block access to the waste hydrogen peroxide filter as the filter is coupled to the separate spaced apart hydrogen peroxide configured vacuum pump, with no access to the waste water channels and waste water storage container. The pump may expel hot air and steam from the sterilization chamber to create optimal conditions for sterilization 426. In an embodiment which employs a sterilization system with one pump for use in both a cycle of steam or vaporized hydrogen peroxide sterilization, the one pump is used in the steam sterilization cycle. In an embodiment, wherein the system includes a plurality of vacuum pumps, wherein each of the pumps is only configured for use with one of the sterilization mediums, the vacuum pump configured for steam sterilization is used in the steam sterilization. The expelled steam and hot air may be directed through a delivery channel, such as a suitable tubing into a cooler for cooling the hot air and steam 428. The cooler may be accessible to the expelled steam and hot air in a steam sterilization cycle. A bypass configured to bypass the cooler may be reversibly blocked. Water and condensation produced by the cooler may be directed to a wastewater storage container 430.

The steam and heat in the sterilization chamber may sterilize the at least one item placed therein 432. After sterilization is complete, the sterilized items may be dried. Drying may be achieved using pulses of air and vacuum. In an embodiment of the hybrid sterilization system which employs a one vacuum pump for use in both a cycle of steam or vaporized hydrogen peroxide sterilization, the one pump is used in the drying stage of the steam sterilization cycle. In an embodiment, wherein the system includes a plurality of vacuum pumps, wherein a pump is only configured for use with one of the sterilization mediums, the vacuum pump configured for steam sterilization is used in the drying stage of the steam sterilization. In some embodiments drying may include heating the sterilization chamber to dry the chamber and its contents. The sterilized, dry items may be removed and stored before use.

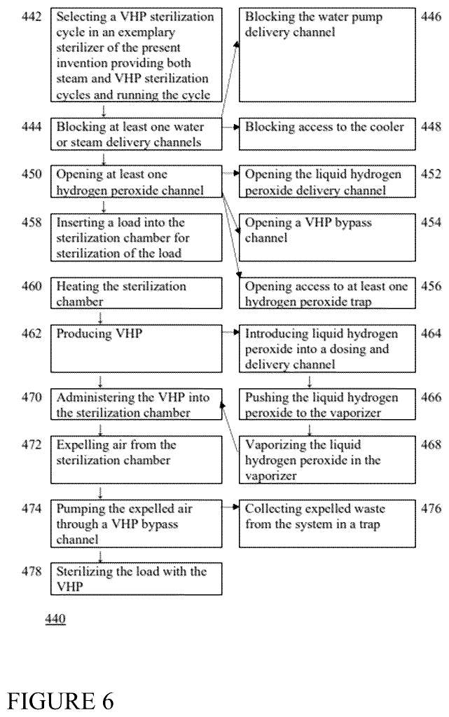

The present invention provides a method of sterilizing an item using vaporized hydrogen peroxide sterilization in a hybrid sterilization system of the present invention providing both steam sterilization cycles and vaporized hydrogen peroxide sterilization cycles 440 as shown schematically in FIG. 6. The order of the steps is not meant to be limiting and any suitable order may be used.

A sterilization cycle may be selected and run. A user may select a vaporized hydrogen peroxide sterilization cycle 442. In one embodiment, the computer may determine a vaporized hydrogen peroxide sterilization cycle for the item for optimal sterilization of that item. Typical items that may be sterilized using vaporized hydrogen peroxide include, but are not limited to phacoemulsification handpieces, posterior segment surgical equipment handpieces, liposuction handpieces, liposuction cannulas, expanders, laparoscopic electrodes, airways, tubing, dental high-speed hand pieces, irrigation lines, cameras, X-Ray accompanying pieces, scanner tips, sensors, dental implant surgery instruments and instruments with tube cavities. When the type of sterilization medium has been chosen as vaporized hydrogen peroxide, the control of the system may block any of the water or steam delivery channels, which may interfere with the vaporized hydrogen peroxide sterilization cycle or be affected by the vaporized hydrogen peroxide sterilization cycle 444.

The control may reconfigure the system to block and open channels according to the chosen cycle. Such reconfiguration for use of the vaporized hydrogen peroxide sterilization cycle may include the following. The water pump delivery channel may be blocked with any suitable access control means configured for reversibly blocking access such as, but not limited to a valve, which can close the channel until needed 446. Access to the cooler may be blocked with a suitable access control means configured for blocking, such as a valve or a tap 448. In an embodiment, wherein the system includes a plurality of vacuum pumps, wherein a pump is only configured for use with one of the sterilization mediums, the vacuum pump configured for sterilization with hydrogen peroxide is used in the vaporized hydrogen peroxide sterilization. Access to the steam vacuum pump may be blocked. Blocking access to the cooler may also block access to the steam vacuum pump which is suitably coupled to the cooler, such as via a distribution channel. The control may open channels needed for sterilization with vaporized hydrogen peroxide 450. A valve may be manipulated to open the liquid hydrogen peroxide delivery channel 452 for providing liquid hydrogen peroxide to the vaporizer. A vaporized hydrogen peroxide bypass channel for bypassing the vaporized hydrogen peroxide sensitive cooler, may be opened by a suitable access control means such as, but not limited to a valve or tap 454. Access may be provided to a trap for absorbing hydrogen peroxide waste 456. The access may be provided by configuring of a suitably positioned access control means. The trap may be a suitable filter, which may be attached adjacent and distal to the outlet of the vacuum pump. In an embodiment, wherein the system includes a plurality of vacuum pumps and a pump is only configured for use with one of the sterilization mediums, the trap may be coupled to the vacuum pump configured for use with hydrogen peroxide. As the trap is only coupled to the vacuum pump configured for use with hydrogen peroxide with no possibility of the waste water channels being exposed to the contents of the trap, the trap may be connected to the outlet of the vacuum pump, such that the access to the trap is unhindered without the need for an access control means.

At least one item may be inserted in any suitable way in the sterilization chamber 458. The at least one item may be sensitive to heat and as such a sterilization cycle with hydrogen peroxide, which is at lower temperatures than steam sterilization may be recommended. The at least one item may be placed on a shelf. The at least one item may be wrapped or unwrapped. In some embodiments the chamber and load are heated pre sterilization. The sterilization chamber may be heated by heating sources, such as a heating jacket disposed about the periphery of the sterilization chamber, a door heater and a back wall heater 460. The pre sterilization heating stage may be for any suitable length of time. The chamber may be heated to a temperature of from about 55° C. to about 57° C. Pre sterilization heating may reduce the amount of condensation of hydrogen peroxide in the sterilization chamber and on the item being sterilized. After the chamber is preheated, the heaters may be used to heat the chamber and load during sterilization. In the sterilization stage the chamber may be heated to a temperature of from about 55° C. to about 57° C.

Vaporized hydrogen peroxide may be produced 462. Liquid hydrogen peroxide may be introduced into the system and provided to the vaporizer in any suitable way 464. In some embodiments a needle with a bore may be attached to an inlet of a hydrogen peroxide inlet delivery channel. The needle may be manipulated to pierce a container of liquid hydrogen peroxide. The container may have a penetrable cover, such as a septum cap for storing the liquid. A dosing mechanism may be employed to supply the correct volume of liquid hydrogen peroxide to the system. The bottle may have a suitable dispenser to dispense a predetermined volume of the liquid hydrogen peroxide. The portion of liquid may be transferred into the delivery channel via the needle. In some embodiments at least two spaced apart valves in the inlet delivery channel may be manipulated to draw the liquid into the delivery channel. The liquid may be exposed to the same low pressure as the sterilization chamber. Manipulation may be by opening and closing of the valves. The liquid may be accommodated between the two valves. The delivery channel may be exposed to a small amount of air at atmospheric pressure by opening of an air valve. This may facilitate pushing of the liquid hydrogen peroxide along the delivery channel to the vaporizer 466. The opening and closing of the valves may be done automatically by the system and controlled by the controller. In one embodiment the valves may be opened and closed manually.

The liquid hydrogen peroxide may be vaporized by the vaporizer 468. The resulting vaporized hydrogen peroxide may be transferred into the sterilization chamber via an inlet in the sterilization chamber 470.

A pump such as a vacuum pump may expel air from the sterilization chamber to create optimal conditions for sterilization 472. In an embodiment which employs a sterilization system with one pump for use in both a cycle of steam or vaporized hydrogen peroxide sterilization, the one pump is used in the vaporized hydrogen peroxide sterilization cycle. In an embodiment, wherein the system includes a plurality of vacuum pumps, wherein a pump is only configured for use with one of the sterilization mediums, the vacuum pump configured for use with vaporized hydrogen peroxide is employed in the vaporized hydrogen peroxide sterilization cycle. The expelled air may be pumped through a delivery channel and via a cooler bypass channel 474. Access to the cooler may be suitably blocked. Condensation, hydrogen peroxide and any byproducts which have been pumped out of the chamber can be trapped by a filter attached in a suitable way, such as to an outlet of the vacuum pump 476.

The vaporized hydrogen peroxide in the sterilization chamber may sterilize the at least one item placed therein 478. After sterilization is complete, the sterilization chamber may be heated to a suitably low temperature of less than about 60° C. to facilitate drying of the chamber and its contents. In some embodiments the sterilization chamber may be heated to a suitably low temperature of less than about 57° C. In some embodiments the sterilized items may be dried by applying pulses of air and vacuum in the chamber. In an embodiment which employs a sterilization system with one vacuum pump for use in both a cycle of steam or vaporized hydrogen peroxide sterilization, the one vacuum pump is used in the drying stage of the vaporized hydrogen peroxide sterilization cycle. In an embodiment, wherein the system includes a plurality of vacuum pumps, wherein a pump is only configured for use with one of the sterilization mediums, the vacuum pump configured for vaporized hydrogen peroxide sterilization is used in the drying stage of the vaporized hydrogen peroxide sterilization cycle. In some embodiments, when the at least one item to be sterilized is unwrapped, the drying may be achieved using only heat. The sterilized, dry items may be removed and stored before use.

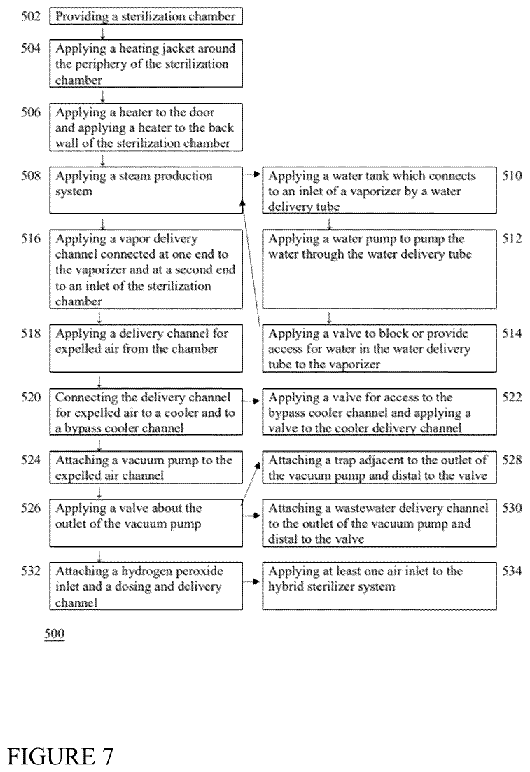

The present invention provides a method of production of a hybrid sterilizer system which can provide both a steam sterilization cycle and a vaporized hydrogen peroxide sterilization cycle 500 as shown schematically in FIG. 7. A sterilization chamber is provided 502. The sterilization chamber may include a door. The sterilization chamber may include at least one inlet for providing the chamber with a sterilization medium. The sterilization chamber may also be configured with at least one outlet for expelling from the chamber a sterilization medium, such as, but not limited to air, steam, hydrogen peroxide vapor, byproducts and mixtures thereof. A heating jacket may be applied around the body of the sterilization chamber 504. A heater may be applied to the door and a heater may be applied to the back wall of the chamber 506.

A steam production system may be applied and attached to the inlet of the sterilization chamber 508. The steam production system may include a water tank. Tubing may connect the water tank to a vaporizer. The tubing may be configured to transport the water 510 out of the water tank. A water pump may be connected to an end of a length of the tubing. The water pump may be spaced apart distal from the water tank 512. Adjacent to the outlet of the water pump a valve may be connected to block or provide flow of the water to the vaporizer through a length of the tubing connecting the outlet of the water pump to the vaporizer 514. The tubing may be connected to an inlet of the vaporizer. The vaporizer may be made from materials, which are resistant to water, heat and hydrogen peroxide. One non-limiting example of a vaporizer is an anodized coated aluminum heat exchanger. One extremity of a steam delivery channel may be connected to the outlet of the vaporizer and may be attached at a second extremity to the inlet of the sterilization chamber 516.

To an outlet of the sterilization chamber may be connected a channel for expelling material out of the sterilization chamber, such as hot air, steam and hydrogen peroxide vapor 518. The expelled air channel may be configured with a bifurcation so that one branch of the expelled channel is connected to a cooler and a second branch of the bifurcation is configured as a bypass channel, which circumvents the cooler 520. Valves may be attached about the two branches to the cooler and bypass channel 522. A valve may be attached to open or close access to the bypass channel. A valve may be attached to open or close access to the cooler inlet. At the outlet of the cooler may be attached one end of an additional length of the expelled air channel. A vacuum pump may be provided, which may be configured or converted to work with both steam and hydrogen peroxide 524. A second end of the additional length of expelled air channel may be attached to an inlet of the vacuum pump. Adjacent and distal to the outlet of the vacuum pump may be attached a valve, such as a three-way valve 526. A trap, such as a filter configured to filter and absorb hydrogen peroxide and other waste products pumped out of the sterilization chamber may be attached adjacent to the pump outlet 528. To the outlet of the vacuum pump may be attached one extremity of a wastewater delivery channel, the other end of the wastewater delivery channel connected to the wastewater container 530. The three-way valve may be configured in relation to the trap and the wastewater storage container so that it can either provide access to the trap or access to the wastewater storage container. A hydrogen peroxide inlet and a dosing and delivery channel may be applied 532.

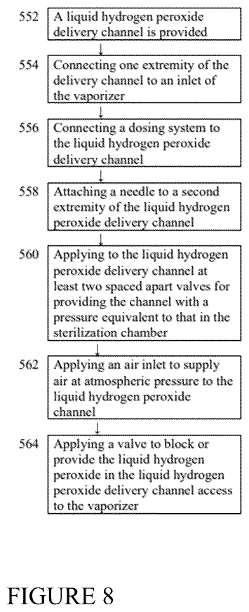

A method for applying the hydrogen peroxide inlet and a dosing and delivery channel is shown schematically in FIG. 8. A liquid hydrogen peroxide delivery channel may be provided 552. The delivery channel may be made from a hydrogen peroxide resistant material such as Teflon. A distal extremity of the delivery channel may be connected to an inlet of the vaporizer 554. The inlet of the vaporizer may have a multi way connection means configured to connect to both the water delivery channel and the liquid hydrogen peroxide delivery channel. A dosing system may be attached to the liquid hydrogen peroxide delivery channel 556. The dosing system may include an injection needle, which may be suitably attached to an inlet at a proximal extremity of the liquid hydrogen peroxide delivery channel 558. The dosing system may further include adjacent and distal to the proximal delivery channel inlet and positioned spaced apart from the proximal inlet, at least two valves 560. The valves may be attached spaced apart and configured for providing in the section of the liquid hydrogen peroxide delivery channel between the two valves, a pressure which is equivalent to the pressure in the sterilization chamber. The two valves and the resulting pressure may be configured to draw a small volume of liquid hydrogen peroxide from a supply of hydrogen peroxide and through the inlet needle. An air inlet with an open and close valve may be provided and attached adjacent to the second valve, and may be configured so that it can be manipulated to supply air at atmospheric pressure 562 to the liquid hydrogen peroxide delivery channel. The pressure difference facilitates propelling the liquid hydrogen peroxide along the delivery channel to the vaporizer and into the sterilization chamber. An access control means configured to reversibly block the hydrogen peroxide inlet and delivery channel may be attached 564. The access control means may be positioned at a spaced apart distance distal from the two dosing valves, attached to a section of the liquid hydrogen peroxide delivery channel disposed between the second dosing valve and the vaporizer.

Referring back to FIG. 7, at least one air inlet, which can be opened and closed may be applied to the system 534. The at least one air inlet may be configured to facilitate controlling the pressure and vacuum in the chamber. The control, electronics, computer, software, screen and other components used by a sterilizer may be applied for the correct working of the system.

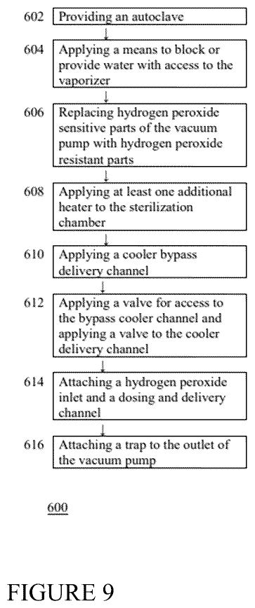

The present invention provides a method of converting an autoclave for steam sterilization to a hybrid sterilizer configured for both steam sterilization and sterilization with vaporized hydrogen peroxide 600 as shown schematically in FIG. 9. An autoclave may be provided 602. The autoclave may feature all the components necessary for steam sterilization. The autoclave may include a sterilization chamber. The sterilization chamber may be made from stainless steel. The sterilization chamber may feature a door for access to the internal cavity of the chamber. The sterilizer may include a heating jacket positioned so that it is wrapped about the periphery of the sterilization chamber. The sterilizer may include a water storage system and a delivery system for delivering a flow of water to a vaporizer. The sterilizer may include a wastewater tank for storing condensed steam pumped out of the chamber and cooled. The autoclave may include a vaporizer for converting the water to steam. The sterilizer may include a delivery channel for delivering steam from the vaporizer into the inlet of the sterilization chamber. The sterilizer may include a hot air and steam delivery channel to transport expelled hot air and steam to a cooler connected to the expelled air delivery channel. The sterilizer may feature a pump to evacuate air from the sterilization chamber and pump the hot air through the cooler. The sterilizer may be connected to a power source to power the sterilizer. A control may be configured to control the sterilization cycle, such as, but not limited to heat, time, pressure, opening and closing of valves, volume of water, volume of steam, pumping and temperature.

An access control means configured to reversibly block or provide water with access to the vaporizer may be added 604. The access control means may be at least one bypass element such as, but not limited to a valve or an equivalent element which can be manipulated automatically or manually. The access control means to block the path of the water may be open to provide water with access to the sterilizer during a steam sterilization cycle and may be closed during a vaporized hydrogen peroxide sterilization cycle. In some embodiments the autoclave includes an access control means, which is disposed between the water pump and the vaporizer and is configured to block or provide water with access to the vaporizer. In such an embodiment the autoclave includes a valve for blocking high pressure from reaching the water pump. This same valve may be used in the converted autoclave eliminating the need to apply an additional access control means to block or provide water to the vaporizer.

Parts of the vacuum pump, which are sensitive to hydrogen peroxide may be replaced with hydrogen peroxide resistant components 606. Hydrogen peroxide sensitive parts may include, but are not limited to inlet and outlet pipe fittings.

At least one additional heater may be applied 608. A heater may be installed in the door of the sterilization chamber. An additional heater may be connected to the opposite back wall of the sterilization chamber. The heaters may be conducive for preventing condensation of hydrogen peroxide in a VPH sterilization cycle.

A cooler bypass delivery channel may be applied 610. The bypass channel may be attached adjacent to the extremities of the cooler to provide a passageway, which circumvents the cooler for hydrogen peroxide expelled from the sterilization chamber. The bypass channel is made from a hydrogen peroxide resistant material, such as, but not limited to Teflon. A component, which can be manipulated to provide access to the bypass channel or block access to it may be applied, such as a valve or tap 612. The bypass channel may be used during a vaporized hydrogen peroxide sterilization cycle. An access control component may also be attached at a suitable distance adjacent to the inlet of the cooler. The access control component may be any element, which by manipulation can either block access to the cooler or provide access to the cooler. Suitable access control components include valves and taps. The valve may be used to block access to the cooler during a vaporized hydrogen peroxide sterilization cycle. The valve may also be used to provide access to the cooler during a steam sterilization cycle.

A hydrogen peroxide inlet and a dosing and delivery channel may be applied 614. The delivery channels are made from a hydrogen peroxide resistant material such as Teflon. One extremity of the delivery channel may be connected to an inlet of the vaporizer. The inlet may have a multi way connection means configured to connect to both the water delivery channel and the liquid hydrogen peroxide delivery channel. A dosing system may be attached to the liquid hydrogen peroxide delivery channel as described hereinabove in FIG. 8.

A trap, such as a filter may be applied adjacent to the outlet of the vacuum pump 616. The filter may be configured to absorb hydrogen peroxide and byproducts pumped out of the chamber. A non-limiting example of a suitable filter is an active carbon filter. Adjacent and distal to the outlet of the vacuum pump and proximal to the trap may be attached a valve, such as a three way valve. The valve may be configured in relation to the trap and a delivery channel connected to the wastewater storage container of the autoclave so that it can either provide access to the trap or access to the wastewater storage container.

The order of the steps is not meant to be limiting and any suitable order may be used.

Reference is made to the following example, which together with the above descriptions illustrates the invention in a non-limiting fashion.

### Example 1

A technician sterilizes surgical scissors. A system of the present invention is used, the system configured for providing the option of using either a sterilization cycle with steam or with vaporized hydrogen peroxide. The technician selects a steam sterilization cycle. The system blocks access to the liquid hydrogen peroxide delivery channel, the cooler bypass channel and the hydrogen peroxide filter. The surgical scissors are placed in the chamber and the scissors are sterilized with steam. When sterilization is complete, the scissors are dried in the chamber. The technician can then take the sterilized surgical scissors out of the sterilization chamber to be reused.

The technician then sterilizes anesthesia tubing, which is heat sensitive. The heat sensitive tubing is placed in the same sterilization chamber previously used for sterilizing the surgical scissors with steam. A vaporized hydrogen peroxide sterilization cycle is selected. The sterilizer system blocks the water delivery channel and access to the cooler. The system opens the cooler bypass channel and provides access to the hydrogen peroxide filter attached to the outlet of the vacuum pump. The system opens the valve to provide liquid hydrogen peroxide in the liquid hydrogen peroxide channel access to the vaporizer. The vaporized hydrogen peroxide sterilization cycle runs and sterilizes the tubing with hydrogen peroxide vapor. When sterilization is complete the tubing is dried in the chamber. The sterilized and dried tubing may then be taken out of the chamber and can be reused or stored until needed.

One skilled in the art can appreciate from the foregoing description that the broad systems, devices, and techniques of the aspects of the present invention can be implemented in a variety of forms. Therefore, while the aspects of this invention have been described in connection with particular examples thereof, the true scope of the aspects of the invention should not be so limited since other modifications will become apparent to the skilled practitioner upon a study of the specification, and following claims.

1. A dual purpose sterilizer system for providing steam sterilization and vaporized hydrogen peroxide sterilization, the sterilizer system comprising:
a sterilization chamber for sterilizing at least one item;
at least one heater for heating the chamber, comprising:
a heater attached to the periphery of the chamber; and
a heater attached to the door of the chamber;
at least one vaporizer configured for vaporizing hydrogen peroxide from a source of hydrogen peroxide and for generating steam from water, the at least one vaporizer for providing vaporized hydrogen peroxide or steam to the chamber;
at least one pump for removing air from the chamber;
at least one delivery system for delivering a flow of the liquid and vaporized hydrogen peroxide or flow of the water and steam in the sterilizer system;
a bypass system for bypassing hydrogen peroxide sensitive components of the sterilizer system; and
a power source for powering the sterilizer system;
a system for providing steam sterilization to the sterilization chamber, comprising:
a source of water;
the at least one delivery system comprising channels for introducing water and for delivering the water and steam in the sterilizer system;
a pump for pumping the water through the channels;
a plurality of valves positioned about the channels, which can be configured to block the channel or provide access to the channel; and
a heat exchanger for cooling the steam and hot air expelled from the chamber by the at least one air removing pump, wherein the expelled steam and hot air are cooled prior to reaching the at least one air removing pump;
wherein the at least one vaporizer is configured for generating steam for providing steam to the chamber; and
a system for providing vaporized hydrogen peroxide sterilization to the sterilization chamber, comprising:
a source of liquid hydrogen peroxide;
the at least one delivery system comprising channels for introducing a dose of liquid hydrogen peroxide into the sterilization system and for delivering the liquid and vaporized hydrogen peroxide in the sterilizer system;
a plurality of valves positioned about the channels, which can be configured to block the channel or provide access to the channel;
wherein the at least one vaporizer is configured to provide vaporized hydrogen peroxide to the sterilization chamber; and
wherein the sterilization chamber is configured for sterilizing the at least one item with vaporized hydrogen peroxide or steam.

2. The sterilizer system of claim 1, comprising a bypass system to bypass at least part of the system for providing vaporized hydrogen peroxide sterilization to the sterilization chamber and a bypass system to bypass at least part of the system for providing steam sterilization to the sterilization chamber.

3. (canceled)

4. The sterilizer system of claim 1
wherein the at least one vaporizer is a vaporizer configured for vaporizing hydrogen peroxide from a source of hydrogen peroxide and for generating steam from water, the vaporizer for providing vaporized hydrogen peroxide or steam to the chamber.

5. (canceled)

6. The sterilizer system of claim 1, wherein the plurality of valves of the system for providing vaporized hydrogen peroxide sterilization to the sterilization chamber comprises:
at least two valves for drawing out a dose of the liquid hydrogen peroxide, by opening and closing of the valves to expose the dose to the pressure of the sterilization chamber.

7. (canceled)

8. The sterilizer system of claim 6, wherein the system for providing vaporized hydrogen peroxide sterilization to the sterilization chamber comprises a bypass system to bypass at least part of the system for providing steam sterilization to the sterilization chamber and wherein the bypass system comprises at least one valve, which can be closed to block at least part of the steam sterilization system and wherein the system comprises at least one access control element to block or provide access to at least part of the at least one delivery system for delivering the liquid hydrogen peroxide.

9. (canceled)

10. (canceled)

11. (canceled)

12. (canceled)

13. The sterilizer system of claim 4 wherein the system for providing steam sterilization to the sterilization chamber comprises at least one access control element to block at least part of the at least one delivery system for delivering the liquid hydrogen peroxide.

14. (canceled)

15. (canceled)

16. The sterilizer system of claim 4, wherein the at least one pump is a vacuum pump configured for use with steam and vaporized hydrogen peroxide.

17. The sterilizer system of claim 1, wherein the at least one heater further comprises:
a heater attached to the back wall of the chamber.

18. (canceled)

19. (canceled)

20. A dual purpose sterilizer system for providing steam sterilization and vaporized hydrogen peroxide sterilization, the sterilizer system comprising:
a sterilization chamber for sterilizing at least one item;
at least one heater for heating the chamber;
at least one vaporizer configured for vaporizing hydrogen peroxide from a source of hydrogen peroxide and for generating steam from water, the at least one vaporizer for providing vaporized hydrogen peroxide or steam to the chamber;
at least one pump for removing air from the chamber;
at least one delivery system for delivering a flow of the liquid and vaporized hydrogen peroxide or flow of the water and steam in the sterilizer system;
a bypass system for bypassing hydrogen peroxide sensitive components of the sterilizer system; and
a power source for powering the sterilizer system;
a system for providing steam sterilization to the sterilization chamber, comprising:
a source of water;
the at least one delivery system comprising channels for introducing water and for delivering the water and steam in the sterilizer system;
a pump for pumping the water through the channels;
a plurality of valves positioned about the channels, which can be configured to block the channel or provide access to the channel; and
a heat exchanger for cooling the steam and hot air expelled from the chamber by the at least one air removing pump, wherein the expelled steam and hot air are cooled prior to reaching the at least one air removing pump;
a heat exchanger bypass, which is configured to bypass the heat exchanger and protect it from hydrogen peroxide during a vaporized hydrogen peroxide sterilization cycle;
wherein the at least one vaporizer is configured for generating steam for providing steam to the chamber; and
a system for providing vaporized hydrogen peroxide sterilization to the sterilization chamber, comprising:
a source of liquid hydrogen peroxide;
the at least one delivery system comprising channels for introducing a dose of liquid hydrogen peroxide into the sterilization system and for delivering the liquid and vaporized hydrogen peroxide in the sterilizer system;
a plurality of valves positioned about the channels, which can be configured to block the channel or provide access to the channel;
wherein the at least one vaporizer is configured to provide vaporized hydrogen peroxide to the sterilization chamber; and
wherein the sterilization chamber is configured for sterilizing the at least one item with vaporized hydrogen peroxide or steam.

21. The sterilizer system of claim 6, further comprising a trap disposed about the outlet of the at least one pump for removing air from the chamber, the trap for filtering odours and remnants of hydrogen peroxide and comprising an access control element to block the filter during a steam sterilization cycle.

22. (canceled)

23. The sterilizer system of claim 1, wherein the at least one vaporizer is a plurality of vaporizers and wherein one vaporizer is configured for vaporizing hydrogen peroxide from a source of hydrogen peroxide and a second vaporizer is configured for generating steam from water.

24. The sterilizer system of claim 4, wherein the at least one pump for removing air from the chamber is a plurality of pumps and wherein one pump is configured to be employed during a sterilization cycle with vaporized hydrogen peroxide and a second pump is configured to be employed during a steam sterilization cycle.

25. (canceled)

26. A method of steam sterilization of at least one item comprising:
inserting at least one item in the sterilization chamber of claim 4;
selecting a steam sterilization cycle;
blocking at least part of the delivery system for delivering a flow of the liquid and vaporized hydrogen peroxide;
heating the sterilization chamber;
removing air from the sterilization chamber with the at least one pump, the at least one pump comprising a vacuum pump;
cooling the removed hot air and steam evacuated from the chamber;
opening the delivery system for facilitating the flow of water in the system to the vaporizer;
pumping water into the at least one vaporizer to produce steam;
introducing the steam into the evacuated sterilization chamber; and
sterilizing the at least one item with the steam.

27. (canceled)

28. (canceled)

29. The method of claim 26, wherein cooling the removed hot air comprises:
providing the expelled air and steam with access to a heat exchanger; and
pumping the hot air and steam through the heat exchanger to cool it; and
wherein providing access to the heat exchanger comprises opening at least one valve for facilitating opening a channel to the heat exchanger.

30. (canceled)

31. The method of claim 26, wherein the at least one pump is a plurality of pumps and wherein a first pump is configured to be employed during a sterilization cycle with vaporized hydrogen peroxide and a second pump is configured to be employed during a steam sterilization cycle and wherein removing air from the sterilization chamber with the at least one pump comprises removing air with the second pump configured to be employed during a steam sterilization cycle.

32. (canceled)

33. A method of sterilization of at least one item with vaporized hydrogen peroxide comprising:
inserting at least one item in the sterilization chamber of claim 4;
selecting a vaporized hydrogen peroxide sterilization cycle;
blocking at least part of the delivery system configured for delivering a flow of water and steam;
heating the sterilization chamber and the at least one item to prevent hydrogen peroxide condensation;
using the bypass system to block and bypass a heat exchanger for cooling air and steam in a steam sterilization cycle;
removing air from the sterilization chamber with the at least one pump, the at least one pump comprising a vacuum pump;
drawing a dose of liquid hydrogen peroxide through and into an inlet of the at least one delivery system for delivering liquid hydrogen peroxide;
facilitating flow of the liquid hydrogen peroxide to the at least one vaporizer;
vaporizing the liquid hydrogen peroxide to produce vaporized hydrogen peroxide;
introducing the vaporized hydrogen peroxide into the chamber; and
sterilizing the at least one item with the vaporized hydrogen peroxide.

34. The method of sterilization of claim 33, wherein the sterilization system comprises at least two valves for drawing out a dose of liquid hydrogen peroxide, and wherein drawing a dose comprises opening and closing of the valves to expose the dose to the pressure of the sterilization chamber.

35. (canceled)

36. The method of claim 33, wherein the sterilization system comprises at least one trap and the method further comprises connecting the trap distally to the outlet of the at least one pump to filter hydrogen peroxide.

37. The method of claim 33, wherein the at least one pump is a plurality of pumps and wherein a first pump is configured to be employed during a sterilization cycle with vaporized hydrogen peroxide and a second pump is configured to be employed during a steam sterilization cycle and wherein removing air from the sterilization chamber with the at least one pump, comprises removing air with the first pump configured to be employed during a vaporized hydrogen peroxide sterilization cycle.

38. (canceled)

39. (canceled)

40. (canceled)

41. (canceled)

42. (canceled)

43. (canceled)

44. The sterilizer system of claim 1, wherein the at least one delivery system for delivering a flow of the liquid and vaporized hydrogen peroxide or flow of the water and steam in the sterilizer system, comprises a channel connected to an inlet of the sterilization chamber for transporting steam or vaporized hydrogen peroxide into the sterilization chamber through the same inlet.
